(19) United States
(12) Patent Application Publication
(10) Pub. No.: US 2024/0293435 A1
(43) Pub. Date: Sep. 5, 2024

(54) Methods and Compositions for Treating Human Disorders Using D-Cycloserine and a Psychedelic Agent

(71) Applicant: William & Mary, Williamsburg, VA (US)

(72) Inventor: Jason P. McDevitt, Williamsburg, VA (US)

(21) Appl. No.: 18/588,947

(22) Filed: Feb. 27, 2024

Related U.S. Application Data
(60) Provisional application No. 63/487,323, filed on Feb. 28, 2023.

(51) Int. Cl. A61K 31/675; A61K 31/405; A61K 31/42

(52) U.S. Cl. CPC A61K 31/675; A61K 31/405; A61K 31/42

(57) ABSTRACT

Methods and pharmaceutical compositions are described for treating a variety of chronic human afflictions including anxiety disorders, eating disorders, chronic pain, depression, addictive disorders, dementia (e.g., Alzheimer's disease, frontotemporal dementia, Lewy body disorder, and vascular dementia), traumatic brain injury (TBI) and mild cognitive impairment. A sub-hallucinogenic dose of a psychedelic tryptamine, including for example psilacetin and psilocybin, along with a low dose of D-cycloserine are administered to human subjects in order to modify neural pathways to treat the chronic affliction.

### CROSS-REFERENCE TO RELATED APPLICATIONS

The present application claims priority under 35 U.S.C. § 119(e) to (i) U.S. Provisional Patent Application No. 63/487,323, filed Feb. 28, 2023. The entire disclosure of this application is incorporated by reference herein.

### BACKGROUND OF THE INVENTION

Psychedelic substances, including naturally occurring psychedelic compounds such as those found in certain mushroom species, have been used by humans for thousands of years.

Recently, there has been a tremendous rebirth in the study of psychedelic compounds for treatment of various human afflictions, particularly depression. The tryptamine psilocybin is often described as the active ingredient in so-called “magic mushrooms”, although depending on the particular psychedelic mushroom species, there can be mixtures of many different psychedelic compounds in psychedelic mushrooms. Depending on the type of psychedelic mushroom, the psilocybin content of the dried mushrooms is often between about 0.25% and 2.0% by dry weight. For example, in Psilocybe cubensis, psilocybin typically comprises between about 0.4% and 1.5% of the dry weight of the mushroom.

Psilocybin and other psychedelic compounds have been proposed and studied for the treatment of a wide range of human afflictions, including anxiety disorders (e.g., obsessive-compulsive disorder, panic disorder, and PTSD), anorexia nervosa, cluster headaches, depression, addiction, and dementia. Some of these studies have yielded very promising results, particularly for the treatment of depression.

In typical clinical studies with psilocybin, psychedelic doses are administered to subjects in the presence of one or more trained personnel, and after being administered psilocybin, the subjects are normally monitored (during the psychedelic trip) for extended periods (e.g., six hours) until the psychedelic effects have worn off. Many people self-medicate using psychedelic mushrooms, and they often utilize the services of a “trip sitter”. While a psychedelic experience can provide significant therapeutic value, this process can require a significant investment of time and resources, and also can be unpredictable and can exacerbate afflictions in some people. It would be advantageous to get the therapeutic benefits of psilocybin or structurally similar psychedelic agents without the side effect of a psychedelic trip.

Low-dose administration of psychedelic compounds is widely practiced as well. This can involve dosing at amounts sufficient to experience either mild effects or no effects at all. In such cases wherein sub-hallucinogenic doses of psychedelic compounds are administered, there is no need for a trip sitter to ensure safety. However, the clinical evidence in support of the efficacy of sub-hallucinogenic dosing is limited, and higher doses have been reported to be more effective, particularly for treatment of depression (Goodwin et al., “Single-Dose Psilocybin for a Treatment-Resistant Episode of Major Depression”, N Engl J Med 2022; 387:1637-48.).

Most psychedelic drugs fall into one of the three families of chemical compounds: tryptamines (e.g., psilocybin), phenethylamines (e.g., mescalin), or lysergamides (e.g., LSD). These psychedelic agents are serotonin receptor agonists, generally with significant activity at serotonin 5-HT2A receptors.

Ketamine is the only psychedelic drug currently approved by FDA (noting some people would argue that ketamine is not a psychedelic). Unlike the aforementioned psychedelic compounds, ketamine's primary mode of activity appears to be as an NMDA receptor antagonist.

Ketamine has been combined with the tuberculosis drug D-cycloserine in clinical trials for treatment of bipolar depression. After initial acute treatment with ketamine, D-cycloserine is administered at high doses in combination with lurasidone. At high doses of greater than 250 mg, DCS is typically an NMDA receptor antagonist (depending on the individual) and often has psychotomimetic side effects. Lurasidone is co-administered to reduce these psychotomimetic side effects.

At low doses (75 mg or lower), the NMDA receptor partial agonist DCS typically increases NMDA receptor transmission, and thus has the opposite effect on NMDA receptor transmission relative to when DCS is administered at high doses.

The processes of learning, memory, and neuroplasticity rely on the mechanism of NMDA receptors. However, overactivation of NMDA receptors can lead to excitotoxicity, which has been implicated in some neurodegenerative disorders such as Alzheimer's disease, Parkinson's disease and Huntington's disease.

DCS has been shown to improve learning and enhance memory in some situations. As a result, DCS has been the subject of many patents and patent applications covering neuropsychiatric disorders including Alzheimer's Disease (see, for example, Cordi, U.S. Pat. No. 5,061,721, and Tsai, U.S. Pat. No. 6,228,875), anxiety disorders (see, for example, Davis, U.S. Pat. No. 7,750,030), and depression (McDevitt, U.S. Pat. No. 8,309,535). In WO2008118785A2, McDevitt et al. describe administering DCS in conjunction with immediate impact treatments for depression such as ketamine or electroconvulsive shock therapy.

DCS has been widely studied in human clinical trials as a potential treatment for dementia conditions including Alzheimer's Disease, showing benefits in at least some of the clinical trials, but not showing significant benefits in others. For example, a study from Tsai et al. concluded that daily administration of 100 mg DCS had a positive benefit on Alzheimer's Disease (Tsai et al., Am J Psychiatry. 1999 March; 156(3): 467-9). In contrast, a multi-center, 410-patient clinical trial conducted by Searle Pharmaceuticals concluded that DCS at up to 50 mg daily dosing did not have a significant benefit for treatment of Alzheimer's Disease (Fakouhi et al., J Geriatr Psychiatry Neurol, October 1995, vol. 8, no. 4, pp. 226-230). A meta-analysis looking at numerous clinical studies with DCS and Alzheimer's Disease concluded that: “The lack of a positive effect of D-cycloserine on cognitive outcomes in controlled clinical trials with statistical power high enough to detect a clinically meaningful effect means that D-cycloserine has no place in the treatment of patients with Alzheimer's disease.” (Laake et al., Cochrane Database Syst Rev. 2002; (2): CD003153).

DCS has shown promise in animal studies for treatment of other forms of dementia. For example, based on rat studies, DCS has been suggested as a candidate for treatment of frontotemporal dementia (Warmus et al., “Tau-Mediated NMDA Receptor Impairment Underlies Dysfunction of a Selectively Vulnerable Network in a Mouse Model of Frontotemporal Dementia”, The Journal of Neuroscience, 3 Dec. 2014, 34(49): 16482-16495) and also dementia related to Parkinson's Disease (Behav. Brain Res. 2011 Jun. 1; 219(2): 280-90). In U.S. Pat. No. 9,877,951, McDevitt describes treatment of dementia by administering DCS on a pulsed basis.

DCS, when administered daily for significant periods, induces physiological changes that reduce its activity. For example, Werner-Seidler and Richardson showed that the well-known ability of DCS to facilitate extinction learning is eliminated in rats if the rats have been exposed to DCS for 15 consecutive days. Moreover, antidepressants can have similar effects in countering the activity of DCS, and daily administration of imipramine for 15 days also blocked the ability of DCS to facilitate extinction learning (Werner-Seidler et al., “Effects of D-Cycloserine on Extinction: Consequences of Prior Exposure to Imipramine”, Biological Psychiatry, Volume 62, Issue 10, 15 Nov. 2007, Pages 1195-1197). Parnas et al. (Neurobiology of Learning and Memory, 83 (2005) 224-231) showed that administering DCS to rats for five consecutive days, followed by a conventional extinction learning paradigm two days later, significantly decreased the ability of DCS to facilitate extinction. In contrast, when DCS was administered for five consecutive days, followed by a conventional extinction learning paradigm 28 days later, the ability of DCS to facilitate extinction was restored. DCS also enhances development of tolerance to other compounds, including ethanol (see Khanna et al., CNS Drug Reviews, Vol. 5, No. 2, pp. 165-176).

Psychedelic compounds, including isolated compounds as well as mixtures from naturally occurring plants, have shown significant promise in treating numerous chronic human health conditions. However, the most promising results have been obtained using doses of psychedelic compounds that induce psychedelic effects. It would be useful to obtain significant therapeutic benefits with psychedelic compounds while using doses too small to induce full psychedelic effects.

### SUMMARY OF THE INVENTION

Methods and pharmaceutical compositions for treating a variety of chronic human afflictions are described herein. A psychedelic tryptamine (or multiple psychedelic tryptamines) and D-cycloserine are administered to patients in need thereof in order to modify neural pathways to treat afflictions including anxiety disorders (e.g., obsessive-compulsive disorder, panic disorder, PTSD, social anxiety disorder), eating disorders (e.g., anorexia nervosa, bulimia), chronic pain, depression, addiction, dementia (e.g., Alzheimer's disease, frontotemporal dementia, Lewy body disorder, and vascular dementia), traumatic brain injury (TBI) and mild cognitive impairment.

In typical embodiments, a psychedelic tryptamine (I), or pharmaceutically acceptable salt thereof, and D-cycloserine, or a pharmaceutically acceptable salt thereof, are administered together as a pharmaceutical composition. In other embodiments, the two active agents are not contained within the same formulation but are administered in separate doses within 30 minutes of each other. In other embodiments, D-cycloserine is administered in conjunction with any psychedelic tryptamine, not limited to the tryptamine (I).

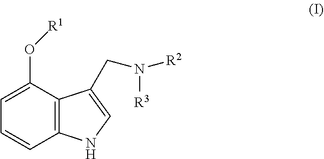

The pharmaceutical compositions of DCS and tryptamine (I) include psilocin prodrugs such as psilocybin and psilacetin, in addition to other tryptamines of formula (I), where R¹ is H, acetyl, C(O)CH₂CH₃, or P(O)(OH)₂, and R² and R³ are independently hydrogen, methyl, ethyl, n-propyl, or isopropyl. These tryptamines are serotonin receptor agonists, and all are 5-HT_{1B} receptor agonists.

A tryptamine (I) is administered along with D-cycloserine (DCS) on a pulsed basis and is administered at sub-hallucinogenic doses. In all embodiments, the psychedelic tryptamine (I) is administered at a dose between about 0.5 mg and about 6 mg. In some embodiments, a mixture of psychedelic tryptamines is utilized (e.g., in a botanical drug preparation comprising dried psychedelic mushrooms), and the active tryptamine (I) is administered at a dose between about 0.5 mg and about 6 mg.

Both DCS and tryptamine (I) rapidly cause habituation, and thus the two active pharmaceutical agents are each administered no more than twice per week, as it is crucial to administer both active ingredients on a pulsed, non-chronic basis.

DCS is an NMDA receptor partial agonist, and is administered at a dose between about 40 mg and about 100 mg. Higher doses of DCS (e.g., 250 mg and above) typically are functional NMDA receptor antagonist doses and thus reduce, rather than increase, NMDA receptor transmission (i.e., at high doses, DCS is an NMDA receptor antagonist).

Importantly, the combination of DCS and the psychedelic tryptamine provides more effective therapeutic outcomes than either active ingredient alone, and in some embodiments, the therapeutic impact of the combined treatment is greater than the additive benefits of either pharmacologic agent alone.

Psychedelic drugs, including the claimed tryptamines and the NMDA receptor antagonist ketamine, have been widely described in recent years as effective treatments for depression. The NMDA receptor antagonist ketamine has been approved by FDA for treatment of depression, and a pharmaceutical combination of lurasidone and high-dose DCS is in Phase Ill human clinical studies for long-term administration after ketamine to treat suicidal depression.

The methods and pharmaceutical combinations described herein are significantly different from the practice and principle of the prior art. Rather than administering a psychedelic tryptamine as the only active pharmaceutical ingredient, or alternatively administering an NMDA receptor antagonist after or in conjunction with a psychedelic drug (e.g., as is done with ketamine followed by DCS/lurasidone), herein we describe administration of (i) a sub-hallucinogenic dose of a psychedelic tryptamine and (ii) a low dose of DCS to increase (rather than decreases) NMDA receptor transmission. This approach promotes neuroplasticity and neurogenesis, and can augment the new learning and neural connections generated by the psychedelic tryptamine.

Relative to treatment with a psychedelic tryptamine alone at sub-hallucinogenic dosages, the combination of a sub-hallucinogenic dosage of psychedelic tryptamine and DCS can provide increased magnitude and duration of benefit. Moreover, by co-administering DCS with a psychedelic tryptamine such as tryptamine (I), sub-hallucinogenic doses of the psychedelic tryptamine can be administered while achieving therapeutic benefits associated with higher doses, thereby simplifying administration of the tryptamine, e.g., eliminating the need for a trip sitter, avoiding extended periods set aside for treatment, reducing costs, reducing side effects, and reducing the likelihood of a bad trip that can exacerbate conditions such as depression, panic, and PTSD. While low-dose administration of psychedelic agents such as psilocybin is widely known and widely practiced (e.g., among users of psychedelic mushrooms, including the use of pulsed, non-daily administration schedules), the method of combining sub-hallucinogenic dosing of psychedelic tryptamines with low doses of DCS to stimulate NMDA receptor transmission is novel.

In some embodiments, the pharmaceutical combination is administered in conjunction with a therapy session, which can involve a therapist or can be computer-based therapy in the absence of a therapist. For example, the pharmaceutical combination can be administered to a human subject within one hour (before or after) of a psychotherapy session. The combination with therapy can improve therapeutic outcomes.

In typical embodiments, the pharmaceutical combination of tryptamine (I) and DCS is administered between about once per week and about twice per week to human subjects afflicted with a chronic disorder such as an anxiety disorder (e.g., obsessive-compulsive disorder, panic disorder, PTSD, social anxiety disorder), an eating disorder, chronic pain, depression, addiction, dementia (e.g., Alzheimer's disease, frontotemporal dementia, Lewy body disorder, and vascular dementia), TBI, and mild cognitive impairment, or combinations thereof. For example, a patient with chronic pain and depression can be treated on a once-weekly basis with a pharmaceutical combination of DCS (75 mg) and psilocybin (3 mg). In another representative embodiment, a patient with depression and mild cognitive impairment is administered a capsule containing 50 mg DCS and 1 mg O-acetylpsilocin (psilacetin) every fourth day for a year. In another representative embodiment, a patient with depression and mild cognitive impairment is administered a capsule comprising a botanical drug combination containing 50 mg DCS and 125 mg of dried Psilocybe cubensis mushrooms every fourth day for a year.

While the methods and pharmaceutical combinations described herein will not cure or necessarily even positively impact every patient, by altering neural connectivity, they can help many people who suffer from these devastating chronic conditions.

### DETAILED DESCRIPTION OF THE INVENTION

As used herein, each of the following terms has the meaning associated with it as described below.

The articles “a” and “an” are used herein to refer to one or to more than one (i.e., to at least one) of the grammatical object of the article. By way of example, “an element” means one element or more than one element.

As used herein, “plurality” means at least two.

As used herein, “FDA” means the United States Food and Drug Administration.

Any ranges cited herein are inclusive, e.g., “between about 0.5 mg and 6 mg” includes compositions of 0.5 mg and 6 mg.

As used herein, a subject is “treated”, or subjected to “treatment”, when an earnest attempt is made to alleviate a medical disorder or disease. For example, a subject can be treated for a disorder by being administered a pharmacologic agent that is intended to alleviate the disorder, irrespective of whether the treatment actually was successful in alleviating the disorder.

As used herein, a disease or disorder or medical affliction is “alleviated” if either the severity of a symptom of the disease or disorder or medical affliction, the frequency with which such a symptom is experienced by a subject, or both, are reduced.

A “subject” of diagnosis or treatment is a human.

A “therapeutic” treatment is a treatment administered to a subject who exhibits signs of pathology for the purpose of diminishing or eliminating those signs.

A “therapeutically effective amount” or “therapeutically effective dose” of the pharmacologic agent is an amount of the pharmacologic agent which typically results in an improved treatment, e.g., a greater therapeutic benefit, or a therapeutic benefit for a longer time, relative to that observed in the absence of administering the pharmacologic agent.

As used herein, the term “therapy” includes terms such as talk therapy, cognitive behavioral therapy, and psychotherapy.

Administering a pharmacotherapeutic agent on a “pulsed basis” is defined as a course of administration wherein over a period of seven days, the pharmacotherapeutic agent is administered to a human subject on two or fewer days, and wherein there is at least one 60-hour period during said seven days during which the pharmacotherapeutic agent is not administered to the subject at all.

As used herein, the term “NMDA receptor” or “NMDA channel” refers to the glutamate receptor channel NMDA subtype (Yamakura and Shimoji (1999) Prog. Neurobiol. 59(3): 279-298). The term “agonist” encompasses any compound that increases the flow of cations through an ionotrophic receptor such as the NMDA receptor, i.e., a channel opener, and which has not been observed to decrease the flow of cations through the same receptor. The term “antagonist” includes any compound that reduces the flow of cations through an ionotropic receptor such as the NMDA receptor, i.e., a channel closer, and which has not been observed to increase the flow of cations through the same receptor. The term “partial agonist” refers to a compound that regulates an allosteric site on an ionotropic receptor, such as the NMDA receptor, to increase or decrease the flux of cations through the ligand-gated channel depending on the presence or absence of the principal site ligand; that is, in the presence or absence of a known endogenous ligand binding to a site on the receptor. In the absence of the principal site ligand, a partial agonist increases the flow of cations through the ligand-gated channel, but at a lower flux than achieved by the principal site ligand. A partial agonist partially opens the receptor channel. In the presence of the principal site ligand, a partial agonist decreases the flow of cations through the ligand-gated channel below the flux normally achieved by the principal site ligand. As used herein, the terms “NMDA receptor agonist”, “NMDA receptor antagonist”, and “NMDA receptor partial agonist” may be alternately referred to as “NMDA agonist”, “NMDA antagonist”, and “NMDA partial antagonist” respectively. Also, “NMDA receptor partial agonist” is intended to be interchangeable with “partial NMDA receptor agonist.”

D-cycloserine, or DCS, refers to the chemical D-cycloserine (CA Index Name: 3-Isoxazolidinone, 4-amino-, (4R)-(9CI); CAS Registry No. 68-41-7), or pharmaceutically acceptable salts thereof, or pharmaceutically acceptable prodrugs thereof. DCS is an FDA (United States Food and Drug Administration)-approved drug for treatment of tuberculosis and is sold in the United States under the trade name Seromycin®. DCS is a structural analog of D-alanine, and is a broad-spectrum antibiotic produced by some strains of Streptomyces orchidaceus and S. garphalus. DCS has antibiotic activity in vitro against growth phase Gram-negative bacteria such as Escherichia coli, some strains of Staphylococcus aureus, and Chlamydia species, among others. The minimum inhibitory concentrations (MIC) in vitro for typical Mycobacterium tuberculosis strains range from about 6-25 μg/mL.

For the treatment of tuberculosis, DCS is generally dosed at 500-1000 mg/day divided twice daily (PDR 1997) with chronic treatment. At a dose of 500 mg/day, serum concentrations of 25-30 μg/ml are generally maintained. Administration of oral capsules of D-cycloserine typically results in peak serum concentrations occurring within 3-8 hours after dosing, with a half-life of 10 hours and primarily renal excretion.

At these typical doses for the treatment of tuberculosis, DCS can give rise to significant neurological side effects in treated subjects. Recorded side effects on chronic dosing schedules (wherein subjects were generally chronically ill with tuberculosis) include drowsiness, depression, headache, confusion, tremor, vertigo, and memory difficulties, paresthesias, and seizure.

As contemplated herein, when combined with tryptamine (I), a therapeutically effective amount of DCS useful for treatment of a human disease or disorder including an anxiety disorder (e.g., obsessive-compulsive disorder, panic disorder, PTSD, social anxiety disorder), an eating disorder, chronic pain, depression, addiction, dementia (e.g., Alzheimer's disease, frontotemporal dementia, Lewy body disorder, and vascular dementia), TBI, and/or mild cognitive impairment is significantly less than the amount of DCS typically used for the treatment of tuberculosis.

When administered to adult human subjects, a therapeutically effective dose of DCS generally comprises a drug formulation (e.g., pill, capsule, tablet) of DCS containing DCS in an amount equal to or greater than 40 mg, and equal to or less than 100 mg. When administered to a subject as disclosed herein, a therapeutically effective serum concentration of DCS is achieved within for example 30 minutes, 1 hour, or even 2 hours after administration.

Serum concentration levels of DCS in subjects to whom DCS is administered are a function of numerous factors, including body weight, metabolism, and the amount of drug ingested. The timing of administration and the therapeutically effective dose of DCS in a given subject will depend on the severity of symptoms, in addition to the age, sex, and size of the subject being treated, among other variables.

DCS is a partial agonist of the NMDA receptor, and doses of greater than 250 mg typically reduce transmission at the NMDA receptor (i.e., serve as functional antagonists of the receptor). Doses of DCS of equal to or greater than 40 mg, and equal to or less than 100 mg, especially doses of between about 40 mg and 75 mg, typically increase NMDA receptor transmission when administered to adult subjects. In order to achieve sufficient concentrations for efficacy, doses of at least 40 mg are needed.

DCS can dimerize upon storage, particularly in the presence of water vapor. Therefore, it can be advantageous to use a prodrug that has enhanced stability during storage and then is transformed to DCS in the body. A particularly suitable prodrug is the condensation product of DCS and acetylacetone, specifically (R)-4-[(1-methyl-3-oxo-1-butenyl)-amino]-3-isoxazolidinone (see Jensen et al., “Use of acetylacetone to prepare a prodrug of cycloserine”, Journal of Medicinal Chemistry (1980) 23 (1), 6-8.)

Psychedelic tryptamines are well-known and increasingly well-studied in the scientific literature. A microdose of psilocybin or psychedelic mushrooms is typically described as a dose that is too small to have perceptual effects. A low dose of these agents typically refers to a dose that gives rise to some perceptual effects (e.g., euphoria, heightened perception) but does not give rise to overt hallucinations. Medium, high, and ultra-high doses give rise to overt hallucinations among other effects. The methods and compositions described herein only utilize microdoses or low doses of psychedelic agents such that side effects are reduced, overt hallucinations do not occur, and external monitoring of the subjects is unnecessary. A “sub-hallucinogenic” dose is defined herein as a dose of a psychedelic tryptamine that is too low to induce overt hallucinations. A hallucination is a perception of visual, auditory, tactile, olfactory, or gustatory experiences without an external stimulus and with a compelling sense of their reality.

While most psychedelic compounds fall into one of the three families of chemical compounds: tryptamines (e.g., psilocybin), phenethylamines (e.g., mescalin), or lysergamides (e.g., LSD), the methods and compositions described herein exclusively utilize certain agents within the tryptamine class of psychedelic agents. Tryptamines have a common chemical structure that is defined by an indole (i.e., a fused benzene and pyrrole ring), and a 2-aminoethyl group at the second carbon of the pyrrole ring. More specifically, the methods require a psychedelic tryptamine, and the pharmaceutical compositions require the psychedelic tryptamine (I).

As defined herein, a psychedelic tryptamine is a tryptamine compound that when administered to humans, can generate hallucinations, distortions of perception, and/or altered states of awareness. While a psychedelic tryptamine can induce psychedelic effects at certain doses, a psychedelic tryptamine can also be administered at doses insufficient to produce overt hallucinations in a human (i.e., sub-hallucinogenic dosing of a psychedelic tryptamine). The methods and compositions described herein exclusively comprise administering sub-hallucinogenic doses of psychedelic tryptamines.

While not all tryptamines of formula (I) have identical effects in the human body (and a given tryptamine can have different effects on different people or on the same person at different times), they are similar in structure and function and can provide therapeutic benefits when paired with DCS according to the methods and compositions described herein.

Suitable tryptamines of formula (I) include psilocin prodrugs such as psilocybin ([3-[2-(dimethylamino)ethyl]-1H-indol-4-yl] dihydrogen phosphate) and O-acetylpsilocin (also referred to as acetylpsilocin, psilacetin, or 4-acetoxy-N,N-dimethyltryptamine). Both psilocybin and O-acetylpsilocin are rapidly converted in the human body to psilocin, which appears to be the primary active agent in the body.

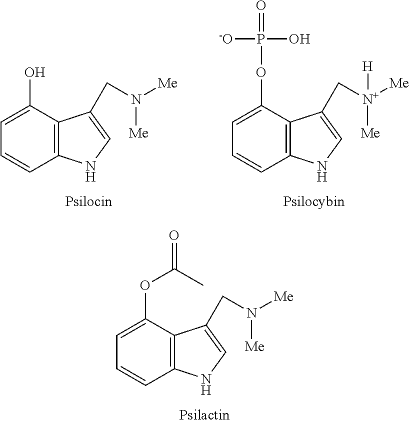

Neuroplasticity is a general term used to describe any change in the way brain cells (neurons) connect to each other, grow, and communicate. Neurogenesis is the specific term for the growth of new brain cells and thus neurogenesis is a subset of neuroplasticity. In addition to neurogenesis, neuroplasticity covers changes in the size of neurons, changes in the connections between neurons, changes in the number or location of connections between neurons, and changes in the strength of those connections. Both DCS and psychedelic tryptamines such as psilocybin can have significant impacts on neuroplasticity, and those effects vary with dosing. DCS can potentially augment beneficial changes in neuroplasticity caused by treatment with psychedelic tryptamines.

Dementia is not a specific disease, but rather an overall term that describes a wide range of symptoms associated with a decline in memory or other skills (e.g., communication and language, ability to focus and pay attention, reasoning and judgment, visual perceptions) severe enough to reduce a person's ability to perform everyday activities. Dementia can be split into two broad categories, i.e., the cortical dementias and the subcortical dementias, based on which part of the brain is affected. Alzheimer's disease accounts for the majority of dementia cases, but there are many other forms of dementia, including but not limited to vascular dementia, frontotemporal dementia, dementia with Lewy bodies, and dementia associated with Parkinson's disease.

Alzheimer's disease is the most common form of dementia. The term “Alzheimer's disease” is widely used as a general term for memory loss and other intellectual abilities serious enough to interfere with daily life. Alzheimer's disease accounts for 60 to 80 percent of dementia cases. Alzheimer's disease is a type of dementia that causes problems with memory, thinking and behavior. Symptoms usually develop slowly and get worse over time, becoming severe enough to interfere with daily tasks.

Vascular dementia is a decline in thinking skills caused by conditions that block or reduce blood flow to the brain, thereby depriving brain cells of vital oxygen and nutrients. In vascular dementia, changes in thinking skills sometimes occur suddenly as a result of strokes that block major brain blood vessels. Thinking problems also may begin as mild changes that worsen gradually as a result of multiple minor strokes or other conditions that affect smaller blood vessels, leading to cumulative damage. The term vascular cognitive impairment is sometimes used instead of the term “vascular dementia”.

Frontotemporal dementia (FTD) is a common form of dementia in humans under the age of 65 years. Variants of FTD include but are not limited to behavioral variant FTD, semantic dementia, and progressive nonfluent aphasia. Behavioral and language manifestations are core features of FTD, and patients have relatively preserved memory, which differs from Alzheimer disease. Common behavioral features include loss of insight, social inappropriateness, and emotional blunting, while common language features are loss of comprehension and object knowledge (semantic dementia), and nonfluent and hesitant speech (progressive nonfluent aphasia). A careful history and physical examination, supplemented as needed by magnetic resonance imaging, is useful in distinguishing FTD from other common forms of dementia, including Alzheimer's disease, dementia with Lewy bodies, and vascular dementia. FTD is characterized by progressive neuronal loss predominantly involving the frontal and/or temporal lobes. Brain atrophy in the frontal and/or temporal lobes is a pathologic feature of the FTD disorders Although no cure for FTD exists, and there are no drugs yet approved by FDA specifically for FTD, symptom management with selective serotonin reuptake inhibitors, antipsychotics, and galantamine has been shown to be beneficial (see Cardarelli et al., “Frontotemporal Dementia: A Review for Primary Care Physicians”, Am. Fam. Physician. 2010 Dec. 1; 82(11): 1372-1377).

Mild cognitive impairment, also known as incipient dementia or age-associated memory impairment, is a brain function syndrome involving the onset and evolution of cognitive impairments beyond those expected based on the age and education of the individual, but which have not yet risen to a sufficient level to significantly interfere with the daily activities of an afflicted individual. It frequently occurs as a transitional stage between the expected cognitive decline of normal aging and the more serious decline of dementia. It can present as problems with memory, language, thinking and judgment that are greater than normal age-related changes. Mild cognitive impairment increases one's likelihood of later progressing to more advanced dementia such as Alzheimer's Disease, but this progression is not a certainty. Herein, mild cognitive impairment is defined as a dementia, although some prior art references refer to it as a pre-dementia.

As defined herein, depression includes three main types: major depressive disorder, dysthymia, and bipolar depression. These particular conditions are defined, for example, in the DSM-V. In some embodiments, this disclosure relates to treating a patient for depression. In a preferred embodiment, such treatment alleviates depression in a subject or patient. Depression is alleviated if either (or both) the severity or frequency of a symptom of the depression is reduced. However, a subject can be treated for depression in accordance with the methods of the invention irrespective of whether the treatment actually was successful in alleviating the depression.

Major depressive disorder is characterized by a state of intense sadness, melancholia, or despair that has advanced to the point of being disruptive to an individual's social functioning and/or activities of daily living.

Persistent depressive disorder, also called dysthymia, is a chronic type of depression in which a person's moods are regularly low for a very long period of time (e.g., two years or more). However, symptoms are not typically as severe as with major depression.

Bipolar depression, also called bipolar disorder and manic depression, causes extreme mood swings that include emotional highs (mania or hypomania) and lows (depression). There are four basic types of bipolar disorder, all of which involve clear changes in mood, energy, and activity levels. These moods range from periods of extremely “up,” elated, and energized behavior (known as manic episodes) to very sad, “down,” or hopeless periods (known as depressive episodes). Less severe manic periods are known as hypomanic episodes. Sometimes a mood episode includes symptoms of both manic and depressive symptoms.

Many individuals suffer from pain, including chronic pain which can have many different causes. Pain ordinarily serves a protective role and then goes away after the healing process is complete. However, sometimes pain persists long after its protective function is no longer necessary, morphing into chronic pain and causing pathological changes to the central nervous system. As used herein, pain means “an unpleasant sensory and emotional experience associated with actual or potential tissue damage or described in terms of such damage”, which is the definition that has been given by the International Association for the Study of Pain. Chronic pain is often associated with other human disorders such as cancer, fibromyalgia, and depression.

Anxiety disorders include, but are not limited to, panic disorder, agoraphobia, social phobia, specific phobia, post-traumatic stress disorder (PTSD), obsessive-compulsive disorder, and generalized anxiety disorder. The disorders contemplated herein are defined in, for example, the DSM-IV (Diagnostic and Statistical Manual of Mental Disorders (4th ed., American Psychiatric Association, Washington D.C., 1994)).

Addictive disorders, also referred to as addiction, include substance-abuse disorders. A substance use disorder is a mental disorder that affects a person's brain and behavior, leading to a person's inability to control their use of substances such as legal or illegal drugs, alcohol, or medications.

Eating disorders are complex mental health conditions that often require the intervention of medical and psychological experts to alter their course. These disorders are described in the American Psychiatric Association's Diagnostic and Statistical Manual of Mental Disorders, fifth edition (DSM-5). Widely known eating disorders include anorexia nervosa, bulimia (bulimia nervosa), and binge eating disorder.

Traumatic brain injury occurs when a sudden, external, physical assault severely damages the brain. It is a broad term describing a wide variety of injuries to the brain, including both closed brain injury and penetrating brain injury. The damage can be confined to one area of the brain or extended to more than one area of the brain, and the severity can range from a mild concussion to a severe injury that results in coma or even death.

According to the methods of the invention, both DCS and a psychedelic tryptamine compound, including but not limited to tryptamines of formula (I), are administered to a subject on a pulsed basis which inhibits development of tolerance. Pulsed administration of DCS and/or the psychedelic tryptamine to a subject means that the compounds are not dosed on a chronic, daily basis, but rather administered on an intermittent basis no more than two days out of every seven days. For example, in any given 7-day cycle, DCS and the psychedelic tryptamine are administered to a human subject on the first day, and subsequently administered no more than one additional time within that seven-day cycle. In one embodiment, the active ingredients could be administered daily for two days, then not administered for the next 5 days, before starting a subsequent cycle. In another embodiment, DCS and the psychedelic tryptamine are administered on a once-weekly basis. In another embodiment, the active ingredients are administered every fourth day for a period of multiple years, for example, for treatment of mild cognitive impairment. Each 7-day cycle does not require consistent treatment. For example, DCS and tryptamine (I) could be administered twice-weekly for the first two-week cycle, then once-weekly for four weeks, then two days in a row followed by 10 days off, then every fifth day. Many other embodiments are contemplated.

In all such embodiments, the key feature of pulsed dosing is that the efficacy of the combined treatment with DCS and the psychedelic tryptamine is maintained because the subject does not develop significant tolerance to either drug. Development of tolerance to a drug can depend on many different factors, and tolerance to different effects of a drug do not necessarily take the same time, and instead can vary enormously (Christiaan H. Vinkers and Berend Olivier, “Mechanisms Underlying Tolerance after Long-Term Benzodiazepine Use: A Future for Subtype-Selective Receptor Modulators?”, Advances in Pharmacological Sciences, Vol. 2012, Article ID 416864, 19 pages, 2012. doi: 10.1155/2012/416864). By administering the active agents on a tolerance-inhibiting basis, rather than daily administrations, development of drug tolerance is reduced, and down-regulation or up-regulation of targeted receptors is less likely. This is important for long-term treatment of chronic conditions in accordance with the methods described herein.

Selective serotonin reuptake inhibitors (“SSRIs”, e.g., fluvoxamine, sertraline, citalopram, escitalopram, paroxetine) and tricyclic antidepressants (“TCAs”, e.g., imipramine, desipramine, nortriptyline, chloripramine) are widely used to treat depression, dementia with co-morbid depression, and also to treat patients with dementia or mild cognitive impairment who have been misdiagnosed as having anxiety disorders. Unfortunately, both TCAs and SSRIs can interfere with the effects of DCS by inducing conformational changes in the NMDA-receptor subunit to which D-cycloserine binds, thereby modulating the effects of DCS treatment (Raabe, R., and Gentile, L. “Antidepressant Interactions with the NMDA NR1-1b Subunit.” Journal of Biophysics 2008 (2008): 474205). Accordingly, in some embodiments of the invention, DCS and a psychedelic tryptamine are administered to subjects provided (i) neither SSRIs nor TCAs have been administered to the subject within a week prior to administration of DCS and a psychedelic tryptamine, and (ii) neither SSRIs nor TCAs are administered to the subject within a week following administration of DCS and a psychedelic tryptamine, irrespective of the duration of treatment with DCS and the psychedelic tryptamine, e.g., whether DCS and the tryptamine are administered for 2 weeks, 2 months, or 2 years.

In one exemplary embodiment, a subject suffering from Alzheimer's Disease self-administers 50 mg DCS and 3 mg psilocybin on a weekly basis. In another embodiment, a subject suffering from Alzheimer's Disease is administered on a once-weekly basis a capsule containing 60 mg DCS and 1 mg psilocybin.

In one exemplary embodiment, a subject afflicted with mild cognitive impairment self-administers a pharmaceutical composition comprising 40 mg DCS and 2 mg acetylpsilocin every Saturday and Sunday for a year. In another embodiment, a subject afflicted with mild cognitive impairment is administered 75 mg of the DCS prodrug (R)-4-[(1-methyl-3-oxo-1-butenyl)-amino]-3-isoxazolidinone and 5 mg psilocybin on a once-weekly basis. In another embodiment, a subject afflicted with mild cognitive impairment self-administers 100 mg of the DCS prodrug (R)-4-[(1-methyl-3-oxo-1-butenyl)-amino]-3-isoxazolidinone and 4 mg of 4-acetyl-diisopropyltryptamine twice weekly on a pre-sleep basis (defined as administering the active ingredient within one hour prior to a subject's planned sleep, e.g., “going to bed”).

In one exemplary embodiment, a subject afflicted with frontotemporal dementia is administered a pill containing 50 mg DCS and 3 mg psilocybin every four days on a pre-sleep basis. In one exemplary embodiment, a subject afflicted with frontotemporal dementia is administered a pill containing 50 mg DCS and 100 mg dried Psilocybe cubensis mushrooms every four days.

In one exemplary embodiment, a subject afflicted with frontotemporal dementia is administered a pill on a once-weekly basis containing 50 mg DCS and 3 mg psilocybin. In one exemplary embodiment, a subject afflicted with Alzheimer's disease is administered a pill on a once-weekly basis containing 50 mg DCS and 3 mg psilocybin. In one exemplary embodiment, a subject afflicted with Alzheimer's disease is administered on a once-weekly basis a first pill containing 50 mg DCS and a second pill comprising a botanical drug containing 150 mg dried Psilocybe azurescens mushrooms, wherein the two pills are administered within a 30-minute period. In one exemplary embodiment, a subject afflicted with Lewy body disorder is administered a pill on a once-weekly basis containing 50 mg DCS and 3 mg psilocybin. In one exemplary embodiment, a subject afflicted with vascular dementia is administered a pill on a once-weekly basis containing 50 mg DCS and 3 mg psilocybin. In one exemplary embodiment, a subject afflicted with chronic pain is administered a pill on a once-weekly basis containing 50 mg DCS and 3 mg psilocybin. In one exemplary embodiment, a subject afflicted with depression is administered a pill on a once-weekly basis containing 50 mg DCS and 3 mg psilocybin. In one exemplary embodiment, a subject afflicted with anorexia nervosa is administered a pill on a once-weekly basis containing 50 mg DCS and 3 mg psilocybin. In one exemplary embodiment, a subject afflicted with social anxiety disorder is administered a pill on a once-weekly basis containing 50 mg DCS and 3 mg psilocybin. In one exemplary embodiment, a subject afflicted with obsessive compulsive disorder is administered a pill on a once-weekly basis containing 50 mg DCS and 3 mg psilocybin. In one exemplary embodiment, a subject afflicted with panic disorder is administered a pill on a once-weekly basis containing 50 mg DCS and 3 mg psilocybin. In one exemplary embodiment, a subject afflicted with PTSD is administered a pill on a once-weekly basis containing 50 mg DCS and 3 mg psilocybin. In one exemplary embodiment, a subject afflicted with mild cognitive impairment is administered a pill on a once-weekly basis containing 50 mg DCS and 3 mg psilocybin. In one exemplary embodiment, a subject afflicted with an addictive disorder is administered a pill on a once-weekly basis containing 50 mg DCS and 3 mg psilocybin. In one exemplary embodiment, a subject suffering from a traumatic brain injury is administered a pill on a once-weekly basis containing 50 mg DCS and 3 mg psilocybin. In one exemplary embodiment, a subject recovering from stroke is administered a pill on a once-weekly basis containing 50 mg DCS and 3 mg psilocybin. In one exemplary embodiment, a subject re-learning how to walk is administered a pill containing 50 mg DCS and 3 mg psilocybin on a once-weekly basis in conjunction with physical therapy sessions.

In one exemplary embodiment, a subject suffering from frontotemporal dementia is administered a pill containing 50 mg DCS and 3 mg O-acetylpsilocin on a twice-weekly basis for 5 years.

In one planned exemplary embodiment, a double-blind, placebo-controlled clinical trial is conducted with the following parameters. Fifty (50) subjects diagnosed with FTD are enrolled and split into a control group (receiving placebo pill) and a treatment group (receiving capsules containing 75 mg DCS and 2 mg O-acetylpsilocin). The subjects take one pill (either placebo or DCS) every week for 26 weeks. Half of the subjects in each group are instructed to take their pill in the morning, and half are instructed to take their pill on a pre-sleep basis, defined as taking the pill within one hour of going to bed for the night. Primary outcomes include change from baseline on Frontotemporal Dementia Rating Scale (FRS), change in Neuropsychiatric Inventory (NPI) [Time Frame: 26 weeks, NPI is a 12-domain caregiver assessment of behavioral disturbances occurring in dementia: delusions, hallucinations, agitation/aggression, depression/dysphoria, anxiety, elation/euphoria, apathy/indifference, disinhibition, irritability/lability, motor disturbance, appetite/eating, nighttime behavior] and Clinical Global Impression of Change (CGIC) [Time Frame: 26 Weeks, a 7-point scale, using a range of responses from 1 (very much improved) through 7 (very much worse), wherein the clinician compares the participant's current condition to the condition at admission to the project]. Secondary outcomes include, for example, metabolic activity in frontal and temporal lobe and change from baseline on Addenbrooke's Cognitive Examination-III (ACE-III).

In one planned exemplary embodiment, a double-blind, placebo-controlled clinical trial is conducted with the following parameters. Ninety subjects diagnosed with treatment-resistant major depression are split into four groups. One group is administered capsules containing 50 mg DCS and 3 mg psilocybin on a twice-weekly basis, with capsules administered every Saturday and Sunday for eight weeks. A second group is administered capsules containing 3 mg psilocybin on a twice-weekly basis, with capsules administered every Saturday and Sunday for eight weeks. A third group is administered capsules containing 50 mg DCS on a twice-weekly basis, with capsules administered every Saturday and Sunday for eight weeks. For all groups, immediately after taking the capsule, the subjects undergo computer-assisted cognitive behavioral therapy for depression. The primary outcome is improvement on the 21-item Hamilton Depression Rating Scale.

In one planned exemplary embodiment, a double-blind, placebo-controlled clinical trial is conducted with the following parameters. Ninety subjects diagnosed with social anxiety disorder are split into four groups. One group is administered capsules containing 40 mg DCS and 2.5 mg acetypsilocin on a weekly basis for eight weeks. A second group is administered placebo capsules every seven days for eight weeks. A third group is administered capsules containing 40 mg DCS and 2.5 mg acetypsilocin on a weekly basis for eight weeks, while also undergoing a computer-assisted cognitive behavioral therapy session for social anxiety disorder in conjunction with each administration of a capsule, with said cognitive behavioral therapy commencing one hour after a capsule is administered. The primary outcome for this study will be the Liebowitz Social Anxiety Scale (LSAS) administered by a blinded independent rater. Other assessments, including physiological, self-report, and behavioral tasks will be used to assess other exploratory variables.

### Formulation of Pharmaceutical Compositions

Pharmaceutical compositions contemplated by the methods and compositions of the invention may be formulated and administered to a subject for treatment of the diseases or afflictions disclosed herein as described below.

In some embodiments, the invention encompasses the preparation and use of pharmaceutical compositions comprising DCS and/or a psychedelic tryptamine as an active ingredient useful for treating an anxiety disorder (e.g., obsessive-compulsive disorder, panic disorder, PTSD, social anxiety disorder), an eating disorder (e.g., anorexia nervosa, bulimia), chronic pain, depression, addiction, dementia (e.g., Alzheimer's disease, frontotemporal dementia, Lewy body disorder, and vascular dementia), TBI, and mild cognitive impairment. Such a pharmaceutical composition may consist of the active ingredient(s) alone, in a form suitable for administration to a subject, or the pharmaceutical composition may comprise the active ingredient(s) and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The compounds of the invention are also useful when formulated as salts. In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, «-ketoglutarate, and α-glycerophosphate. Suitable acid addition salts of inorganic acids may also be formed, including hydrochloride, sulfate, nitrate, bicarbonate, and carbonate salts. Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

The descriptions of pharmaceutical compositions provided herein are directed to pharmaceutical compositions which are suitable for ethical administration to humans.

Pharmaceutical compositions that are useful in the methods of the invention may be prepared, packaged, or sold in formulations suitable for oral, rectal, vaginal, parenteral, sublingual, topical, pulmonary, intranasal, buccal, ophthalmic, intrathecal or another route of administration. Other contemplated formulations include projected nanoparticles, liposomal preparations, resealed erythrocytes containing the active ingredient, and immunologically based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

The therapeutically effective dose of the pharmacologic agent can be administered using any medically acceptable mode of administration. Although the skilled artisan would contemplate any of the modes of administration known to one of ordinary skill, preferably the pharmacologic agent is administered according to the recommended mode of administration, for example, the mode of administration listed on the package insert of a commercially available agent.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated, including coating using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl parahydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of a dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue, including intramuscular, subcutaneous, intravenous, and intradermal injection. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intravenous, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form—for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butanediol, for example. Other acceptable diluents and solvents include, but are not limited to, Ringer's solution, isotonic sodium chloride solution, and fixed oils such as synthetic mono or di-glycerides. Other parenterally administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, PA, which is incorporated herein by reference.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. In one packaging embodiment, a suitable multi-dose unit is a blister-pack having between four and ten doses of DCS and tryptamine (I). For example, a 8-pack of capsules comprising DCS and tryptamine (I) may be suitably prescribed to a subject.

### Dosage

For treating an anxiety disorder (e.g., obsessive-compulsive disorder, panic disorder, PTSD, social anxiety disorder), an eating disorder (e.g., anorexia nervosa, bulimia), chronic pain, depression, addiction, dementia (e.g., Alzheimer's disease, frontotemporal dementia, Lewy body disorder, and vascular dementia), TBI, and mild cognitive impairment, DCS dosage levels administered to adult humans can be between about 40 mg and 100 mg per day, while tryptamine (I) dosage levels can be between about 0.5 mg and 6 mg per day. The therapeutically effective dose of DCS and tryptamine (I) can be administered using any medically acceptable mode of administration. Although the skilled artisan would contemplate any of the modes of administration known to one of ordinary skill, preferably the pharmacologic agent is administered according to the recommended mode of administration, for example, the mode of administration listed on the package insert of a commercially available agent.

In one embodiment, tablets comprising a pharmaceutical composition of DCS and psilacetin useful for treating, for example, major depressive disorder can be made by: (1) mixing 500 g DCS, 50 g psilacetin, 600 g lactose, and 700 g starch, then adding 300 g of wet starch and mixing to homogeneity; (2) grounding the resultant mass while wet, passing through a 15 mesh sieve, and mixing with 50 g talc, 20 g magnesium stearate, and 30 g silicon dioxide; and (3) compressing the mixture in a rotary compressor and tableting to provide 10,000 tablets each weighing 225 mg and comprising 50 mg DCS, 5 mg psilacetin, 60 mg lactose, 100 mg starch, 5 mg talc, 2 mg magnesium stearate, and 3 mg silicon dioxide. Smaller tablets can be made from the same mixture.

### INCORPORATION BY REFERENCE

All publications, patents, and patent applications cited herein are hereby expressly incorporated by reference in their entirety and for all purposes to the same extent as if each was so individually denoted.

### EQUIVALENTS

While specific embodiments of the subject invention have been discussed, the above specification is illustrative and not restrictive. Many variations of the invention will become apparent to those skilled in the art upon review of this specification. Contemplated equivalents of the methods of treating anxiety related disorders disclosed here include administering fast acting compositions which otherwise correspond thereto, and which have the same general properties thereof, wherein one or more simple variations of substituents or components are made which do not adversely affect the characteristics of the methods and compositions of interest. The full scope of the invention should be determined by reference to the claims, along with their full scope of equivalents, and the specification, along with such variations.

1. A pharmaceutical composition useful for the treatment of a human disease comprising: (i) D-cycloserine, or pharmaceutically acceptable salt thereof, and (ii) a psychedelic tryptamine compound of formula (I); or pharmaceutically acceptable salt thereof, wherein R¹ is H, C(O)CH₃, C(O)CH₂CH₃, or P(O)(OH)₂, and R² and R³ are independently hydrogen, methyl, ethyl, n-propyl, or isopropyl; 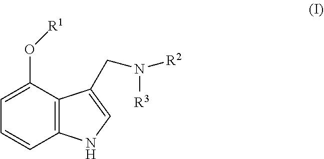
wherein said composition comprises between about 40 mg and about 100 mg D-cycloserine, or pharmaceutically acceptable salt thereof; and
wherein said composition comprises between about 0.5 mg and about 6 mg of said psychedelic tryptamine compound, or pharmaceutically acceptable salt thereof.

2. The pharmaceutical composition of claim 1, wherein said human disease is selected from the group consisting of an anxiety disorder, an eating disorder, chronic pain, depression, addiction, Alzheimer's disease, frontotemporal dementia, Lewy body disorder, vascular dementia, traumatic brain injury, and mild cognitive impairment.

3. The pharmaceutical composition of claim 1, wherein said psychedelic tryptamine compound is a psilocin prodrug.

4. The pharmaceutical composition of claim 3, wherein said psilocin prodrug is selected from the group consisting of psilocybin and psilacetin.

5. The pharmaceutical composition of claim 1, wherein said composition comprises at least five different psychedelic tryptamine compounds of formula (I), and wherein said composition comprises a total of between about 0.5 mg and about 6 mg of psychedelic tryptamine compounds of formula (I).

6. A method for treating a human affliction comprising administering to a human subject (i) D-cycloserine, or pharmaceutically acceptable salt thereof, and (ii) a psychedelic tryptamine compound, or pharmaceutically acceptable salt thereof;
wherein said D-cycloserine, or pharmaceutically acceptable salt thereof, is administered at a dosage of between about 40 mg and about 100 mg;
wherein said psychedelic tryptamine compound, or pharmaceutically acceptable salt thereof, is administered at a dosage of between about 0.5 mg and about 6 mg;
wherein said dosage of said psychedelic tryptamine compound does not induce overt hallucinogenic effects;
wherein said human affliction is selected from the group consisting of an anxiety disorder, an eating disorder, addiction, depression, a psychiatric disorder, Alzheimer's disease, frontotemporal dementia, traumatic brain injury, and mild cognitive impairment;
wherein said psychedelic tryptamine compound, or pharmaceutically acceptable salt thereof, and said D-cycloserine, or pharmaceutically acceptable salt thereof, are both administered within a 30-minute time period; and
wherein neither said psychedelic tryptamine or pharmaceutically acceptable salt thereof, nor said D-cycloserine or pharmaceutically acceptable salt thereof, is administered more than twice within a seven-day period.

7. The method of claim 6, wherein said psychedelic tryptamine compound is a compound of formula (I), or pharmaceutically acceptable salt thereof, 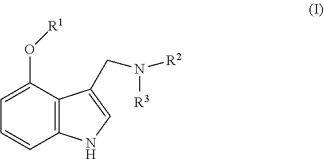
wherein R¹ is H, C(O)CH₃, C(O)CH₂CH₃, or P(O)(OH)₂, and R² and R³ are independently hydrogen, methyl, ethyl, n-propyl, or isopropyl.

8. The method of claim 6, wherein said D-cycloserine or pharmaceutically acceptable salt thereof, and said psychedelic tryptamine compound or pharmaceutically acceptable salt thereof, are administered to said human subject within two hours of commencement of a therapy session involving said subject.

9. The method of claim 6, wherein said D-cycloserine or pharmaceutically acceptable salt thereof, and said psychedelic tryptamine compound or pharmaceutically acceptable salt thereof, are both administered to said human subject multiple times, wherein the interval between successive administerings is at least 64 hours.

10. The method of claim 6, wherein said administering results in improvement within 24 hours of a symptom of a human affliction selected from the group consisting of an anxiety disorder, an eating disorder, chronic pain, depression, addiction, Alzheimer's disease, frontotemporal dementia, Lewy body disorder, vascular dementia, traumatic brain injury, and mild cognitive impairment.

11. The method of claim 6, wherein said administering results in alleviation within one week of at least one symptom of a human affliction selected from the group consisting of an anxiety disorder, an eating disorder, chronic pain, depression, addiction, Alzheimer's disease, frontotemporal dementia, Lewy body disorder, vascular dementia, traumatic brain injury, and mild cognitive impairment.

12. The method of claim 6, wherein at least one symptom of a human affliction selected from the group consisting of an anxiety disorder, an eating disorder, chronic pain, depression, addiction, Alzheimer's disease, frontotemporal dementia, Lewy body disorder, vascular dementia, traumatic brain injury, and mild cognitive impairment is alleviated for a period of at least month after said administering.

13. The method of claim 6, wherein said psychedelic tryptamine compound, or pharmaceutically acceptable salt thereof, is a single psychedelic tryptamine compound.

14. The method of claim 6, wherein said psychedelic tryptamine compound, or pharmaceutically acceptable salt thereof, comprises at least five different psychedelic tryptamine compounds.
